(19) United States
(12) Patent Application Publication
(10) Pub. No.: US 2022/0304840 A1
(43) Pub. Date: Sep. 29, 2022

(54) ORTHOPEDIC DEVICES AND SYSTEMS

(71) Applicant: Ahmed Radwan, Swansea, IL (US)

(72) Inventor: Ahmed Radwan, Swansea, IL (US)

(21) Appl. No.: 17/213,652

(22) Filed: Mar. 26, 2021

(51) Int. Cl. A61F 5/01

(52) U.S. Cl. CPC A61F 5/0127; A61F 2005/0153

(57) ABSTRACT

An orthopedic device for treating musculoskeletal disorders of the lower limb. The device includes a leg portion, a foot portion, and a toe portion. The leg portion is rotationally coupled to the foot portion at an ankle joint. The toe portion is rotationally coupled to the foot portion at a toe joint. The foot portion is selectively positionable relative to the leg portion at the ankle joint such that the foot portion may be arranged in a plurality of an ankle angles including plantarflexed and dorsiflexed positions. The toe portion is selectively positionable relative to the foot portion at the toe joint such that the toe portion may be arranged in a plurality of toe angles relative to the foot portion including plantarflexed and dorsiflexed positions.

### FIELD OF THE DISCLOSURE

The field of the disclosure relates generally to devices and systems used for the treatment of musculoskeletal disorders of the lower limb, and more particularly to, an orthopedic device used to facilitate exercising and actively stretching the plantar fascia and the Achilles tendon.

### BACKGROUND

Various orthopedic devices have been used for the treatment of musculoskeletal disorders of the lower limb, including for example, Achilles tendonitis, Plantar fasciitis, and muscle fatigue. Plantar fasciitis is the strain and inflammation of the plantar fascia connecting the calcaneus bone and the metatarsals, resulting in pain and discomfort at the bottom of the foot. Achilles tendinitis is typically caused by an overstretching or overuse of the tendon connecting the calf muscles to the calcaneus, resulting in pain and swelling in the heel and along the rear of the leg. At least some known orthopedic devices attempt to alleviate pain and discomfort associated with these disorders by isolating movement of the foot relative to the lower leg, through splinting for example, to stretch and to prevent contracture of soft tissue structures, i.e., tendons, ligaments, musculature, and fascia, within the foot. For example, at least some known orthopedic devices passively stretch the Achilles tendon and the plantar fascia by positioning the foot in a fixed dorsiflexed position wherein the forward portion of the foot is flexed upwardly such that the toes are positioned closer to an anterior side of the lower leg. Arranging the foot in such a fixed position maintains the fascia and the Achilles tendon in a stretched position, wherein the fascia and Achilles tendon are each elongated.

Proprioceptive neuromuscular facilitation, also referred to as neuromuscular stretching or active stretching, has been shown to enhance active and passive ranges of motion. This treatment includes iteratively placing a muscle and/or tendon in a stretched position with simultaneous isometric contraction of the muscle, followed by a period of relaxation of the stretched muscle and/or tendon. At least some known devices passively retain the foot in a fixed dorsiflexed position and as such, may be generally categorized as splints or braces. Moreover, at least some of such devices are recommended to be worn primarily while the user is sleeping. In such devices, developmental stretch is achieved by positioning and securing the foot in discrete, predetermined fixed angles of dorsiflexed positions. Notably, because the foot is secured in a fixed position, the wearer cannot selectively reposition (e.g., flex or extend) to reposition their foot in other angles of dorsiflexed and/or plantarflexed using muscle activation to control the amount of stretch and/or to selectively stretch and relax the soft tissues structures of the lower limb. Over time, isolated movement of muscle and/or tendon may actually start to weaken the strength of the muscle or tendon.

As such, at least some other treatment devices, known as exercise devices, include resistance bands that are oriented to oppose a user's muscle forces. Although such devices enable a user to actively stretch and strengthen their muscle and/or tendon, such devices do not provide support to the wearer and do not provide controlled rotation of the ankle and/or toes.

Accordingly, a need exists for devices and systems that facilitate alleviating pain and discomfort associated with lower limb musculoskeletal disorders including Achilles tendonitis, Plantar fasciitis, and muscle fatigue, without requiring prolonged isolation of the muscle and/or tendon.

This section is intended to introduce the reader to various aspects of art that may be related to various aspects of the disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

### SUMMARY

In one aspect, an orthopedic device including a leg portion, a foot portion, and a leg portion is provided. The leg portion includes a leg shell including an upper end and a lower end. The leg shell at least partially defines a leg portion cavity sized to receive at least a portion of a user's lower leg. The leg portion includes a leg axis extending between the upper end and the lower end. The foot portion includes a foot shell including a heel end and a toe end. The foot shell at least partially defines a foot portion cavity sized to receive at least a portion of a user's foot. The foot portion includes a foot axis extending between the heel end and the toe end. The foot portion is rotationally coupled to the leg portion at an ankle joint. An ankle angle defined between the foot axis and the leg axis, is selectively positionable in at least one of an acute ankle angle and an obtuse ankle angle. The toe portion includes a toe shell including a first end and a second end. The toe shell at least partially defines a toe portion cavity sized to receive at least a portion of a user's toes. The toe portion includes a toe axis. The toe portion is rotationally coupled to the foot portion at a toe joint such that a toe angle define between the foot axis and toe axis may be selectively positioned in at least one of an acute toe angle and an obtuse toe angle.

Another aspect of the present disclosure is directed to a system for exercising the lower limb. The system includes a leg portion sized to receive at least a portion of the user's leg, a foot portion sized to receive at least a portion of the user's foot, and a toe portion sized to receive at least a portion of the user's toes. The leg portion includes a leg axis. The foot portion includes a foot axis and is rotationally coupled to the leg portion such that an ankle angle between the foot axis and the leg axis may be selectively positionable in a plurality of ankle angles such that the foot portion may be arranged in at least one of plantarflexed position and dorsiflexed position with respect to the leg portion. The toe portion includes a toe axis and the toe portion is rotationally coupled to the foot portion such that a toe angle between the foot axis and the toe axis may be selectively positionable in a plurality of toe angles such that the toe portion may be arranged in at least one of plantarflexed position and dorsiflexed position with respect to the foot portion.

Yet another aspect of the presented disclosure is directed towards a method of fabricating an orthopedic device. The method includes providing a leg portion that includes a leg shell having an upper end, an opposite lower end, and a leg axis extending between the upper and lower ends. Providing a foot portion including a foot shell including a heel end, an opposite toe end, and a foot axis extending between the heel and toe ends. Providing a toe portion that includes a toe shell including a first end, an opposite second end, and a toe axis extending between said first and second ends. Rotationally coupling the leg portion to the foot portion at an ankle joint such that an ankle angle defined between the foot portion axis and the leg portion axis is selectively positionable in one of an acute ankle angle and an obtuse ankle angle. Rotationally coupling the toe portion to the leg portion at a toe joint such that a toe angle defined between the foot portion axis and toe portion axis is selectively positionable in one of an acute toe angle and an obtuse toe angle.

Various refinements exist of the features noted in relation to the above-mentioned aspects of the present disclosure. Further features may also be incorporated in the above-mentioned aspects of the present disclosure as well. These refinements and additional features may exist individually or in any combination. For example, various features discussed below in relation to any of the illustrated embodiments of the present disclosure may be incorporated into any of the above-described aspects of the present disclosure, alone or in any combination.

### BRIEF DESCRIPTION OF THE DRAWINGS

FIG. 1 is a perspective view of an exemplary orthopedic device including a leg portion, a foot portion, and a toe portion worn by a user;

FIG. 2 is a perspective view of another embodiment of an orthopedic device;

FIG. 3 is a side view illustrating a plurality of exemplary ankle positions of the foot portion relative to the leg portion that may be possible with any of the orthopedic devices described herein;

FIG. 4 is a side view illustrating a plurality of exemplary positions of the toe portion relative to the foot portion that may be possible with any of the orthopedic devices described herein;

FIG. 5 is a perspective view of another embodiment of an orthopedic device including an exemplary resistance system;

FIG. 6 is a perspective view of a further embodiment of an orthopedic device including the resistance system shown in FIG. 5 and an exemplary nerve stimulation system; and

FIG. 7 is a perspective view of yet another embodiment of an orthopedic device including an exemplary massage system.

Corresponding reference characters indicate corresponding parts throughout the drawings.

### DETAILED DESCRIPTION

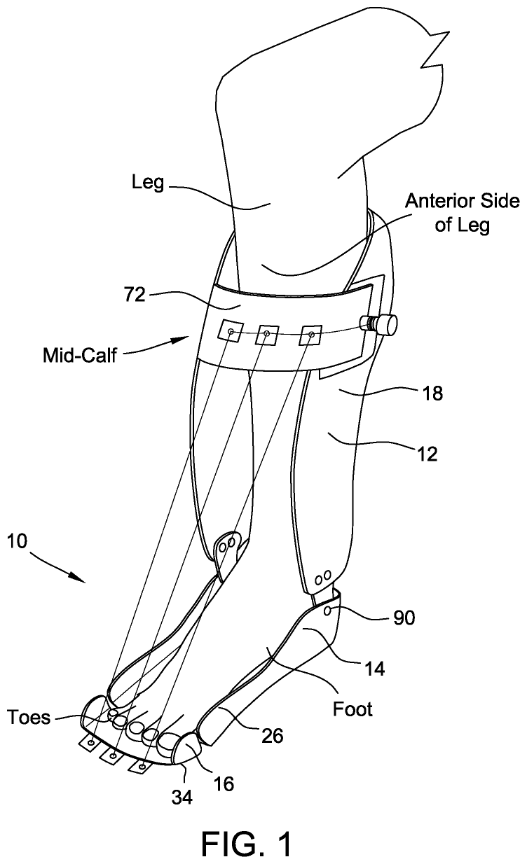

FIG. 1 is a perspective view of an exemplary orthopedic device 10 worn by a user to facilitate treatment of lower limb disorders. In exemplary embodiment, device 10 is an orthopedic device that includes a first portion 12, also referred to herein as a leg portion 12, a second portion 14, also referred to herein as a foot portion 14, and a third portion 16, also referred to herein as a toe portion 16. More specifically, in exemplary embodiment, portions 12, 14, and 16 cooperate to define a “boot” such that foot portion 14 is coupled between leg and toe portions, 12 and 16, respectively. In the exemplary embodiments, device 10 may be worn to facilitate treatment of lower limb disorders, including but not limited to, Achilles tendonitis, Plantar fasciitis, and/or muscle fatigue.

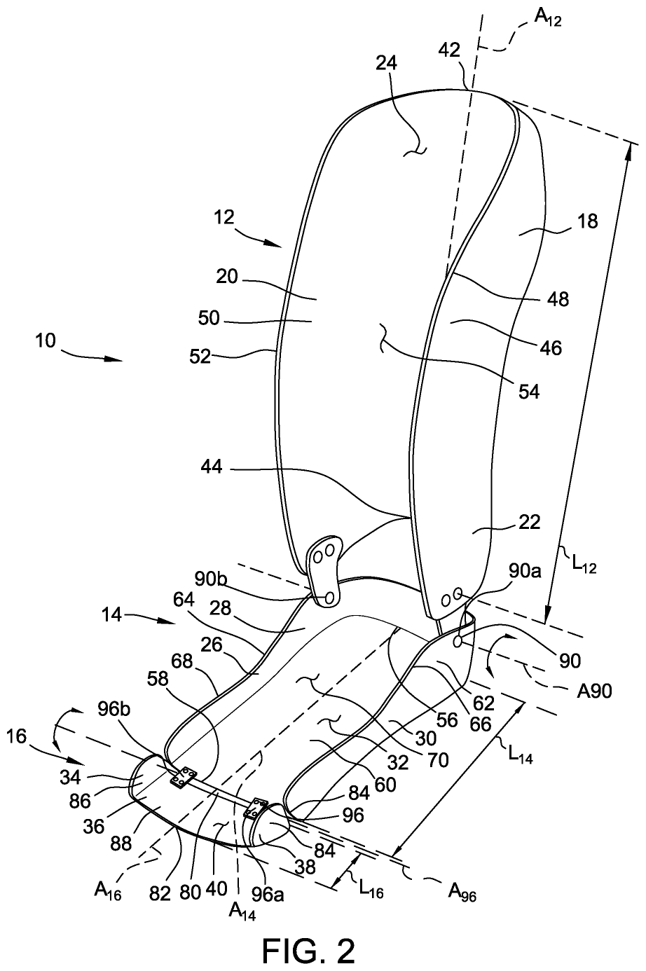

FIG. 2 is a perspective view of exemplary orthopedic device 10. In exemplary embodiment, leg portion 12 includes a leg shell 18 including a leg inner surface 20 and an opposite leg outer surface 22. Leg inner surface 20 partially defines an inner boundary of a leg portion cavity 24 that is sized and shaped to receive at least a portion of a user's lower leg, i.e, user's calf or cnemis, therein. Foot portion 14 includes a foot shell 26 including a foot inner surface 28 and an opposite foot outer surface 30. Foot inner surface 28 partially defines an inner boundary of a foot portion cavity 32 that is sized and shaped to receive at least a portion of a user's foot therein. Toe portion 16 includes a toe shell 34 including a toe inner surface 36 and an opposite toe outer surface 38. Toe inner surface 36 defines a boundary of a toe portion cavity 40 that is sized and shaped receive at least a portion of a user's toes therein. Accordingly, a user may wear device 10, similar to wearing a conventional orthopedic boot, by inserting their lower limb, i.e., their lower leg, foot, and associated toes into each of respective leg, foot, and toe portion cavities 24, 32, and 40, as shown in FIG. 1.

Leg portion 12 includes an upper end 42 and a lower end 44. Device 10 is oriented such that lower end 44 is adjacent to foot portion 14 and upper end 42 is spaced a distance L₁₂ from lower end 44. Distance L₁₂, also known as leg length, is variably selected depending on a length (not shown) of the user's or wearer's lower leg. In exemplary embodiment, an axis of symmetry A₁₂ extends between upper end 42 and lower end 44.

Leg portion 12 includes a medial side 46 including a medial edge 48, and an opposite lateral side 50 including a lateral edge 52. Edges 48 and 52 define, at least partially, a leg opening 54 that is sized and shaped to enable a user to insert their leg through opening 54 such that their leg may be positioned, as described in more detail below, within leg portion cavity 24. When device 10 is worn by a user leg axis A₁₂ may be generally aligned with and/or is substantially parallel to an anatomical axis (not shown) extending through user's lower leg, e.g., a tibia axis. Additionally, during use of device 10, medial edge 48 is oriented adjacent to a medial side of user's leg and lateral edge 52 is oriented adjacent to a lateral side of user's leg, and at least a portion of an anterior side of the user's leg may be exposed from leg opening 54.

Foot portion 14 includes a heel end 56 and a toe end 58 that is a distance L₁₄ from heel end 56. Distance L₁₄, also known as foot length, is variably selected depending on a length (not shown) of user's foot. Heel end 56 is arranged in proximity to leg portion 12 lower end 44 and an axis of symmetry A₁₄ extends between heel end 56 and toe end 58. Foot portion 14 includes a foot base 60, a foot medial side 62 and an opposite foot lateral side 64. Foot medial side 62 and foot lateral side 64 extend substantially perpendicularly from foot base 60. Medial and lateral foot sides 62 and 64 have a medial foot edge 66 and a lateral foot edge 68, respectively, which defines a foot opening 70. Foot opening 70 is sized and shaped such that a user may insert their foot through foot opening 70 such that their foot may be positioned, as described in more detail below, within foot portion cavity 32. When device 10 is worn by a user, axis A₁₄ may be generally aligned with and/or substantially parallel to an anatomical axis (not shown) of foot, e.g., an axis extending through the midline of user's foot. Additionally, during use of device 10, the bottom or lower surface of the user's foot may rest on foot base 60 and foot medial side 62 may be adjacent to a medial side of user's foot. Likewise, foot lateral side 64 is adjacent to a lateral size of user's foot and at least a portion of top of user's foot may be exposed from foot opening 70.

Toe portion 16, includes a first toe end 80 and a second toe end 82. First toe end 80 is adjacent to toe end 58 of the foot portion 14, such that first toe end 80 is spaced a distance L₁₆ from second toe end 82. Distance L₁₆, also known as the toe length, is variably selected depending on a length (not shown) of the user's toes. In the exemplary embodiment, an axis of symmetry A₁₆ extends between first toe end 80 and second toe end 82. Toe portion 16 includes a medial toe side 84 and a lateral toes side 86 that are arranged on each side of a toe plate 88. During use of device 10, axis A₁₆ may be aligned with an anatomical axis (not shown) of a user's toes. Additionally, during use of device 10, user's toes may rest on toe plate 88 and medial toe side 84 may be adjacent to medial side of a user's toes, i.e., hallux, and likewise, lateral toe side 86 is adjacent to lateral side of user's toes, i.e., outermost toe. Additionally, the top of user's toes may be exposed from toe portion cavity 40.

In some embodiments, leg length, foot length, and toe lengths, respective distances L₁₂, L₁₄, and L₁₆, may be customizable, i.e., variably selected, to correspond to the lengths of a user's leg, foot, and toes, respectively, or alternatively, these lengths may be selected based on anthropometric data for lower limb segments.

With reference again to FIG. 1, in the exemplary embodiment, leg portion 12 includes only one leg strap 72. Alternatively, leg portion 12 may include more than one leg strap 72. In the exemplary embodiment, leg strap 72 extends across leg opening 54 and may engage with an anterior side of user's leg to secure and to retain user's leg within leg portion cavity 24. Leg strap 72 is selectively coupled to at least one of medial side 46 and/or lateral side 50 such that a user may selectively couple and/or uncouple leg strap 72 across leg opening 54. In the exemplary embodiment, leg strap 72 is selectively coupled to medial side 46 and is coupled to lateral side 50. Leg strap 72 and outer leg surface 22 includes any suitable attachment mechanisms used to selectively couple leg strap 72 to outer leg surface 22. Suitable attachment mechanisms may include, for example and without limitation, VELCRO®, clasps, fasteners, and the like. In some exemplary embodiments, foot portion 14 may include a foot strap (not shown) that is selectively coupled to at least one of medial or lateral sides 62, 64 and may be extended across foot opening 70. Foot strap may engage with a portion of user's foot to aid in retention of user's foot within foot portion cavity 32. Leg strap 72 and/or foot strap may be made of an elastic material allowing a user to selectively adjust the tension in the strap thereby enabling the user to adjust the fit of device 10.

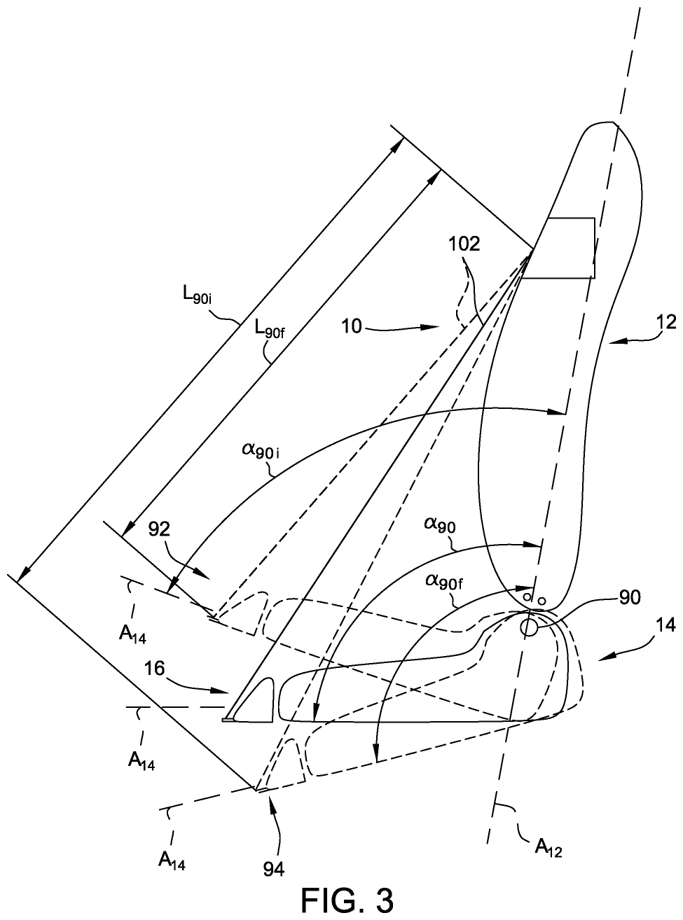

FIG. 3 is a side view illustrating a plurality of exemplary ankle positions of any of orthopedic devices 10. In the exemplary embodiment, leg portion 12 is rotationally coupled to foot portion 14 at an ankle joint 90. Ankle joint 90 enables rotation of foot portion 14 relative to leg portion 12 about an ankle axis A₉₀. Ankle joint 90 includes any suitable cylindrical joint, e.g., a hinge. For example, in the exemplary embodiment, ankle joint 90 includes a medial ankle hinge 90a and a lateral ankle hinge 90b. An ankle angle α₉₀, defined between leg axis A₁₂ and foot axis A₁₄, may be selectively orientated by selectively rotating foot portion 14 and/or leg portion 12 about ankle joint 90. Ankle angle α₉₀ may be selectively positioned in an acute angle, wherein foot portion 14 is in a dorsiflexed position 92 relative to leg portion 12. Additionally, ankle angle α₉₀ may be selectively positioned in an obtuse angle, wherein foot portion 14 is in a plantarflexed position 94 relative to the leg portion 12. Ankle angle α₉₀ may be selectively positioned by plantarflexion motion of foot portion 14 which is the action of extending foot portion 14 which increases ankle angle α₉₀. Foot portion 14 may also be positioned by dorsiflexion motion of foot portion 14 which is the action of flexing foot portion 14 which decrease ankle angle α₉₀. Plantarflexion and dorsiflexion are motions which are used position the ankle angle α₉₀ regardless of if the ankle angle α₉₀ is in a plantarflexed or dorsiflexed position.

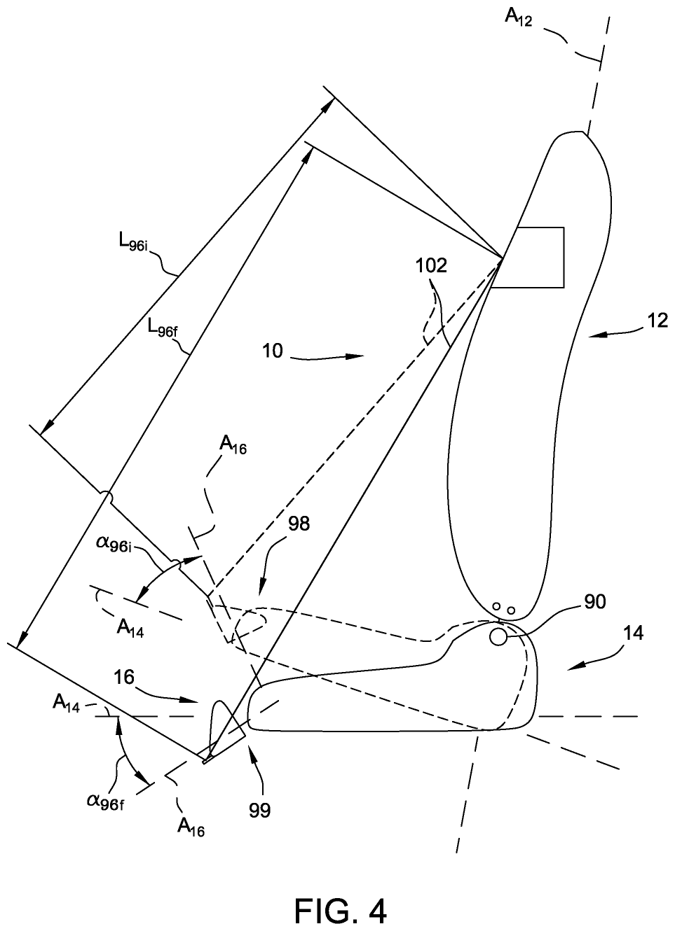

FIG. 4 is a side view illustrating a plurality of exemplary toe positions of any of orthopedic devices 10. Foot portion 14 is rotationally coupled to toe portion 16 at a toe joint 96. Toe joint 96 enables rotation of toe portion 16 relative to foot portion 14 about a toe axis A₉₆. Toe joint 96 includes any suitable cylindrical joint, e.g., a hinge. In the exemplary embodiment, toe joint 96 includes a medial toe hinge 96a and a lateral toe hinge 96b. A toe angle α₉₆ defined between foot axis A₁₄ and toe axis A₁₆, may be selectively orientated by selectively rotating toe portion 16 about toe joint 96 relative to foot portion 14. For example, toe angle α₉₆ may be selectively positioned in an acute angle, i.e., wherein toe portion 16 is in a dorsiflexed position 98 relative to foot portion 14. Additionally, and/or alternatively, toe angle α₉₆ may be selectively positioned in an obtuse angle, i.e., wherein toe portion 16 is in a plantarflexed position 99 relative to foot portion 14. Toe angle α₉₆ may be selectively positioned by plantarflexion motion of toe portion 16 which is the action of extending toe portion 14 which increases toe angle α₉₆. Toe angle α₉₆ may be selectively positioned by dorsiflexion motion of toe portion 16 which is the action of flexing toe portion 16 which decreases toe angle α₉₆. Plantarflexion and dorsiflexion movements may be used to selectively reposition the toe angle α₉₆ regardless of whether the toe angle α₉₆ is in a plantarflexed position or a dorsiflexed position.

Ankle joint 90 and toe joint 96 enables leg portion 12, foot portion 14, and toe portion 16 to be selectively arranged in a plurality of different positions. For example, a user may selectively position ankle angle α₉₀ in an acute angle such that foot portion 14 is in dorsiflexed position 92 relative to leg portion 12 while, simultaneously arranging toe angle α₉₆ in an obtuse angle such that toe portion 16 is in plantarflexed position 99 relative to foot portion 14. Alternatively and/or additionally, a user may selectively arrange both ankle angle α₉₀ and toe angle α₉₆ in an acute angle such that foot portion 14 is in dorsiflexed position 92 relative to the leg portion 12 and toe portion 16 is in dorsiflexed position 98 relative to the foot portion 14.

In some embodiments, device 10 includes an ankle locking mechanism (not shown) used to secure ankle joint 90 in a fixed position. Locking mechanism may be used to secure the ankle joint 90 at any suitable ankle angle that enables a user to walk while wearing device 10. For example, locking mechanism may be used to selectively secure ankle joint 90 such that ankle angle α₉₀ is fixed at approximately 90° . In some exemplary embodiments, device 10 includes a toe locking mechanism (not shown) capable of locking toe joint 96, thereby fixing toe angle α₉₆. Ankle and toe locking mechanisms may include any suitable mechanisms, for example and without limitation, a pin, ratchet hinge, and the like.

In an alternative embodiment, ankle locking mechanism and/or toe locking mechanism may include a spring retractable pin (not shown) including a pull ring (not shown). Angle joint 90 and/or toe joint 96 may also include engagement features (not shown) that are oriented such that when spring retractable pin engages engagement features, ankle joint 90 and/or toe joint 96 are locked. The ankle locking mechanism and/or toe locking mechanism may include a plurality of engagement features oriented to enable the locking mechanisms to lock ankle joint 90 in plurality of ankle angle α₉₀ or to lock toe joint 96 in a plurality of toe angles α₉₆. In some embodiments, leg portion 12, foot portion 14, and toe portion 16 may include secondary engagement features (not shown). The spring retractable pin may engage the engagement features and/or the secondary engagement features to cause leg portion 12 to be locked relative to foot portion 14. Likewise, foot portion 14 and toe portion 16 may include secondary engagement features that are oriented such that when the spring retractable pin engages the engagement features and/or the secondary engagement features, foot portion 14 is locked relative to the toe portion 16. Additionally, locking at least one of ankle joint 90 and/or toe joint 96 enables a user to walk while wearing device 10. In some exemplary embodiments, foot outer surface 30 and/or toe outer surface 38 may include traction features (not shown) that provide a gripping surface between device 10 and the ground. The locking mechanism may also be used to lock or secure the ankle joint 90 at any angle, e.g., an acute angle or an obtuse angle, to facilitate stretching.

In some embodiments, at least one of leg, foot, and toe inner surfaces 20, 28, and 36, respectively, is selectively coupled to at least one cushion (not shown). Cushion enhances comfort to the user and may be used to customize the fit during wear and use of device 10. In some embodiments, a thickness or hardness of the cushion may be varied, such as possible with an inflatable cushion. Cushion is generally sized and shaped to conform to the shape of leg, foot, and toe inner surfaces 20, 28, and 36, respectively. Cushion and foot, leg, and toe inner surfaces 20, 28, and 36, respectively, may include suitable fasteners, such as VELCRO®, clasps, fasteners, and the like, that enable the cushion to be selectively coupled and uncoupled from device 10. Any of leg shell 18, foot shell 26, and/or toe shell 34 may be made of a hard material, such as plastic, while the cushion is fabricated from a softer material such as silicone, foam (e.g., polyurethane foam) and/or the like. In some embodiments, the cushion may include a pneumatically actuated bag (not shown). Moreover, a hand pump (not shown) may be used to selectively inflate bags to facilitate customizing and/or tightening the fit of device 10 about the user's lower leg, foot, and toes. Additionally, and/or alternatively, a hand pump may be used to selectively deflate bags to facilitate customizing or loosening the fit of device 10 about a user's lower leg.

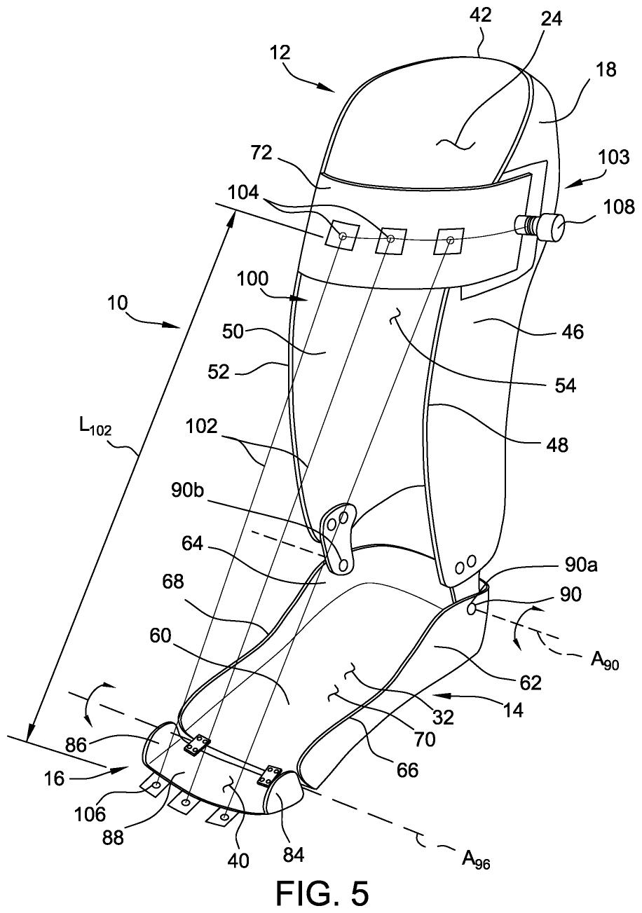

FIG. 5 is a perspective view of another exemplary embodiment of orthopedic device 10 including an exemplary resistance system 100. In the exemplary embodiment, resistance system 100 includes at least one resistance band 102 including a first end 104 and a second end 106. First end 104 is coupled to either leg portion 12 and/or to leg strap 72. Second end 106 is coupled to foot portion 14 and/or to toe portion 16. More specifically, in the exemplary embodiment, first end 104 is coupled at a first attachment location 103 on leg strap 72, and second end 106 is coupled to toe portion 16. First attachment location 103 is approximately halfway between upper and lower ends 42 and 44, respectively. In some embodiments, first attachment location 103 may be substantially aligned with a mid-calf of a user wearing device 10, see FIG. 1. In the exemplary embodiment, resistance band 102 is fabricated from an elastic material that has a desired band stiffness and a predefined free length, i.e., an unloaded length. Elongation of resistance band 102 from its unloaded length results in resistance band 102 exerting a contraction force directed along resistance band 102 between first and second ends 104 and 106, respectively. In some embodiments, band second end 106 is coupled to toe portion 16. Alternatively, and/or additionally, in some embodiments second end 106 is coupled to foot portion 14, for example, to foot medial side 62 and/or to foot lateral side 64. Accordingly, the resistance band 102 resists rotations of ankle joint 90 and/or toe joint 96. Specifically, resistance band 102 facilitates resisting an increase in ankle angle α₉₀ and/or the toe angle α₉₆.

In the exemplary embodiment, band stiffness, e.g., the elastic stiffness of the band 102, and/or its resistance to stretching (elongating) under a load, is variably adjustable. In the exemplary embodiment, at least a portion of resistance band 102 is coupled to a tensioning knob 108 to enable a user to variably adjust a resistance of device 10. More specifically, in the exemplary embodiment, knob 108 is rotationally coupled to leg portion 12 and first end 104 of resistance band 102 is coupled to knob 108. In other embodiments, first end 104 and knob 108 are coupled to leg strap 72. Alternatively, knob 108 may be rotationally coupled to foot portion 14 and second end 106 of resistance band 102 coupled to knob 108. Rotations of knob 108 facilitates increasing band stiffness. Generally, increases in band stiffness require a greater force to stretch or elongate resistance band 102. In the exemplary embodiment, a user may selectively adjust band stiffness by rotating tensioning knob 108 clockwise and/or counterclockwise to selectively increase and/or selectively decrease band stiffness. Alternatively, and/or additionally, band stiffness may be selectively adjustable using any other suitable mechanisms, for example, but not limited to a turn buckle mechanism, a ratchet mechanism, a series of zippers, and/or any other device that facilitates a user selectively adjust the band resistance.

Resistance system 100 includes any suitable number of resistance bands 102 that provide a desired amount of resistance to rotation of ankle joint 90 and/or toe joint 96. More specifically, resistance bands 102 provide resistance in rotating ankle angle α₉₀ or toe angle α₉₀ from a first angle, e.g., a dorsiflexed position, to a second angle that is greater than first angle, e.g., a planter-flexed position. In some exemplary embodiments, resistance band 102 may extend between medial side 46 and foot medial side 62. Alternatively, and/or additionally, resistance band 102 may extend between lateral side 50 and foot lateral side 64. In addition, in some embodiments, resistance bands 102 may extend between leg strap 72 and toe end 58 of foot portion 14. Alternatively, or in addition, resistance band 102 may extend between the leg strap 72 and toe portion 16. In some embodiments, second end 106 of resistance bands 102 may include an attachment feature (not shown) oriented such that second end 106 may be selectively coupled to any location on foot portion 14 and toe portion 16. Known attachment features may include clasp, clips, carabiners, and/or any other feature that enables resistance system 100 to function as described herein. Foot portion 14 and toe portion 16 may include a plurality of engagement features, such as, but not limited to, openings, hooks, clasps, and/or the like, such that an attachment feature associated with second end 106 may be selectively coupled to engagement features associated with foot portion 14 and/or toe portion 16. Accordingly, a user may selectively adjust the position and alignment of resistance bands 102. For example, a user may adjust or readjust the position of resistance band 102 using the attachment features to selectively couple and/or uncouple resistance band 102 to foot portion 14 and/or toe portion 16. A user may adjust or readjust the position and/or alignment of resistance band 102 to accommodate various user's foot and toes shapes and sizes, including for example, users with bunions, bunionette, and/or arthritis, may selectively reposition resistance band 102 such that resistance band 102 is prevented from inadvertently rubbing against selected portions of the user's leg, foot, and/or toes.

Individual resistance bands 102 may be individually coupled to a respective tensioning knob 108, such that a band stiffness of each resistance band 102 may be selectively adjusted independently of other bands 102. Additionally, and/or alternatively, a plurality of resistance bands 102 may be coupled in a group to a single tensioning knob 108 and the band stiffness of that grouping of bands 102 may be adjusted simultaneously.

A length L₁₂₀ of each resistance band 102 increases, stretches, or elongates, with increases in angle ankle α₉₀ and/or increases in toe angle α₉₆. For example, in reference to FIG. 3, in embodiments wherein band second end 106 is coupled to foot portion 14 (alternatively, band second end 106 is coupled to the toe portion 16 and toe joint 96 is locked), when ankle angle α₉₀ is positioned at a first ankle angle α₉₀ᵢ, (e.g., foot portion 14 is in a dorsiflexed position 92 relative to leg portion 12) resistance band 102 is a first length L₉₀ᵢ. When ankle angle α₉₀ is positioned at a second ankle angle α_{90f} (e.g., foot portion 14 is in a plantarflexed position 94 relative to the leg portion 12) that is greater than first ankle angle α₉₀ᵢ, resistance band 102 is a second length L_{90f}. The second length L_{90f} is longer than the first length L₉₀ᵢ. First ankle angle α₉₆ᵢ, may be an acute angle and second ankle α_{90f} may be an obtuse angle. Additionally, and/or alternatively, first ankle angle α₉₀ᵢ, and second ankle angle α_{90f} may both be an acute angle wherein foot portion 14 is in a dorsiflexed position 92 relative to leg portion 12, and second ankle angle α_{90f} is positioned at a larger acute angle than first ankle angle α₉₀ᵢ, Additionally, both the first ankle angle α₉₀ᵢ, and second ankle angle α_{90f} may both be an obtuse angle wherein foot portion 14 is in a plantarflexed position 94 relative to the leg portion 12 and second ankle angle α_{90f} is positioned at a larger obtuse angle than first ankle angle α₉₀ᵢ,

Similarly, in embodiments wherein band second end 106 is coupled to toe portion 16, when toe angle α₉₆ is positioned at the first toe angle α₉₆i (e.g., toe portion 16 is in a dorsiflexed position 98 relative to foot portion 14), resistance band 102 is at the first length L₉₆ᵢ, as shown in FIG. 4. When toe angle α₉₆ is positioned at a second toe angle α₉₆f (e.g., toe portion 16 is in a plantarflexed position 99 relative to foot portion 14) that is greater than the first toe angle α₉₆i, each resistance band 102 is at the second length L_{96f} that is longer than the first length L₉₆ᵢ. First toe angle, α₉₆ᵢ may be an acute angle and second α_{96f} may be an obtuse angle. Additionally, and/or alternatively, first toe angle α₉₆ᵢ and second toe angle α_{96f} may both be positioned at an acute angle wherein toe portion 16 is in a dorsiflexed position 98 relative to foot portion 14, and second toe angle α_{96f} is positioned at a larger acute angle than first toe angle α_{96i.} Additionally, both first toe angle α₉₆ᵢ and second toe angle α_{96f} may be positioned at an obtuse angle wherein toe portion 16 is in a plantarflexed position 99 relative to foot portion 14 and second toe angle α_{96f} is positioned at a larger obtuse angle than first toe angle α₉₆ᵢ.

The band stiffness may be selected such that, under passive ankle flexion, i.e., under no muscle activation, foot portion 14 and/or toe portion 16, may be oriented in dorsiflexed position 92, 98 wherein a stretch is induced to soft tissue structures of a user's lower leg, foot, and/or toes. In this passive position, each resistance band 102 has a length L₁₀₂ that is longer than its unloaded length. During use, a user may secure toe joint 96, e.g., using locking mechanism, at any position relative to device 10 such that each resistance band 102 having its second end 106 coupled to toe portion 16, provides resistance to rotation of ankle joint 90, as opposed to providing resistance to rotation of toe joint 96. Additionally, ankle joint 90 may be secured in position, e.g., using the locking mechanism, to isolate resistance of resistance band 102 to toe joint 96.

A wearer may use muscle activation to selectively position ankle angle α₉₀ and/or toe angle α₉₆ (e.g., flexion and/or extension of foot and/or toes). A user may selectively flex their foot and/or toes to decrease ankle angle α₉₀ and/or toe angle α₉₆. A user may selectively extend their foot and/or toes to increase the ankle angle α₉₀ and/or toe angle α₉₆. Muscle activation may be used to counter the resistance of each resistance band 102 to selectively position the ankle angle α₉₀ and/or toe angle α₉₆ or muscle activation can be used to maintain ankle angle α₉₀ and/or toe angle α₉₆ in a fixed position. Accordingly, device 10, including resistance system 100, is enabled to provide resistance to isotonic contractions of a muscle, in which the muscle changes lengths, for both concentric contractions (muscle contractions in which a muscle shortens while generating a force) and eccentric contractions (muscle contractions in which a muscle elongates while generating a muscle force). Additionally, resistance system 100 provides resistance for isometric contractions in which a muscle generates a force without changing the length of the muscle.

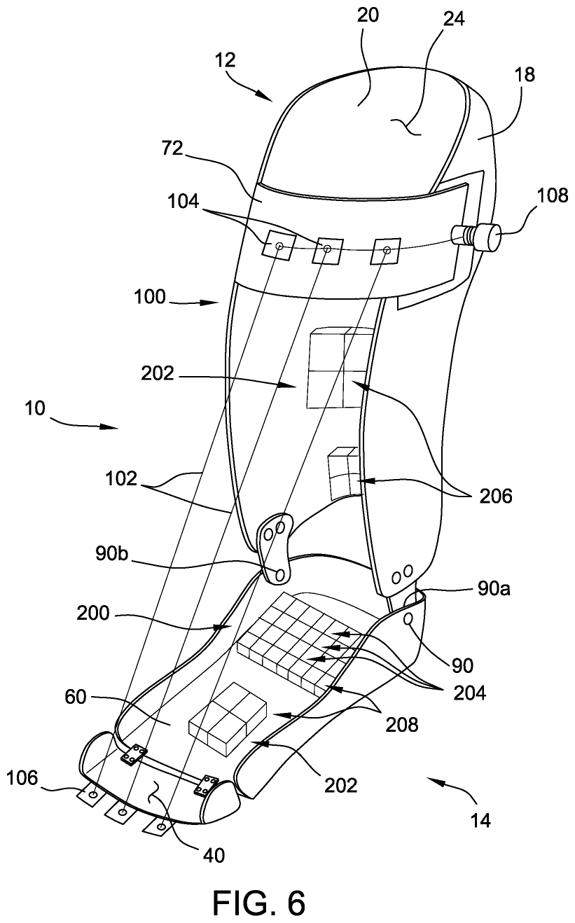

FIG. 6 is a perspective view of a further embodiment of an orthopedic device 10 including resistance system 100 and a nerve stimulation system 200. Nerve stimulation system 200 includes at least one nerve pad 202. Each nerve pad 202 includes a plurality of transcutaneous electrical nerve stimulation units 204, also referred to as TENS unit 204. In the exemplary embodiment, the plurality of TENS units 204 are arranged in a grid-like pattern extending across pad 202, for example, in rows and columns. Alternatively, TENS units 204 may be arranged in any other suitable pattern on pad 202.

In the exemplary embodiment, device 10 includes at least one leg pad 206 coupled to leg portion 12 and at least one foot pad 208 coupled to foot portion 14. Each TENS units 204 on leg pad 206 are coupled to leg inner surface 20 and are biased such that at each leg pad 206 and each TENS units 204 remains in contact a wearer's leg. Likewise, foot pad 208 is coupled to foot inner surface 28 such that at least a portion of foot pad 208 and each TENS units 204 remains in contact with a user's foot.

Each TENS unit 204 is coupled to leg and foot inner surfaces 20 and 28 with a suitable biasing mechanism (not shown) that biases TENS units 204 to maintain contact with user's leg and foot during use of device 10. More specifically, the biasing enables each TENS unit 204 to flex inwardly and/or to extend outwardly relative to adjacent units 204 and relative to leg portion 12 and/or foot portion 14 such that contact is maintained with the user's leg and/or foot. Additionally, and/or alternatively, units 204 may flex side-to-side relative to adjacent units 204, e.g., a portion of unit 204 may move towards or away from adjacent units to ensure that contact is maintained with leg and/or foot. The biasing may be accomplished using any known biasing mechanism including, but not limited to, memory materials, springs, magnets, and or displaceable gels.

In an alternative embodiment, TENS unit 204 may include a plurality of cylindrical openings that each contain a rod (not shown) therein. In such embodiments, each rod includes a tip (not shown) that transmits electrical nerve stimulation to a user wearing device 10. Each rod is translatable relative to the cylindrical opening, and each tip may extend outwardly from the cylindrical opening. The biasing mechanism may include a spring, that is also contained in the cylindrical opening. The spring forces the rod upwardly relative to the cylindrical opening, thus forcing the tip out of the cylindrical opening and into engagement with the user's leg, foot, or toes.

In other alternative embodiments, the TENS unit 204 may be a commercially-available TENs system that is used to treat nerve related pain conditions via electrical stimulations.

In some embodiments, pads 202 are coupled to leg portion 12 and/or foot portion 14 such that pads 202 may be selectively attached and/or detached from the respective leg portion 12 and/or foot portion 14. For example, in one embodiment, a user may selectively reposition the pads 202 to a plurality of different positions on either leg portion 12 and/or foot portion 14. Accordingly, a user may selectively position pad 202 to facilitate nerve stimulation to a selected target area on user's leg and/or foot. For example, pads 202 may be positioned to deliver electrical nerve stimulation to the Achilles tendon or to a ball or mid region of a base of user's foot.

Pads 202 may be operably connected to a battery supply (not shown) and a controller (not shown). Controller includes suitable components enabling a user to turn on and/or off pads 202, or to adjust the intensity of electrical stimulation of pads 202.

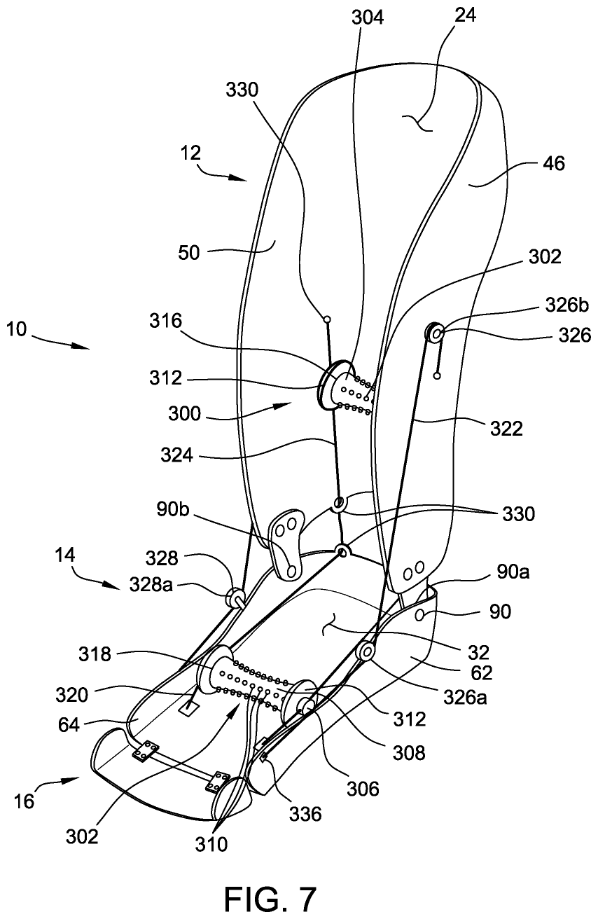

FIG. 7 is a perspective view of another embodiment of orthopedic device 10 including an exemplary massage system 300. In the exemplary embodiment, massage system 300 includes at least one massage roller 302. Massage roller 302 includes an outer roller 304 that is rotationally coupled to a shaft 306. Outer roller 304 includes a roller surface 308 that includes a plurality of protrusions 310 extending outwardly from roller surface 308 to facilitate massaging of a wearer's foot and/or leg during use of device 10. Massage roller 302 includes a pair of wheels 312 coupled to shaft 306 that enable massage roller 302 to roll and translate relative to device 10, as will be described further herein.

In the exemplary embodiment, massager roller 302 includes a leg massager 316 and a foot massager 318. Foot massager 318 is adjacent to foot portion 14 and may roll and/or translate relative to foot portion 14 in a direction substantially parallel to foot axis A₁₄. Leg massager 316 is adjacent to leg portion 12 and may roll and/or translate relative to leg portion 12 in a direction substantially parallel to leg axis A₁₂. Wheels 312 of foot massager 318 roll against foot base 60, and wheels 312 of leg massager 316 roll against leg inner surface 20. Shaft 306 of foot massager 318 is coupled to foot base 60 via at least one biasing element 320. Biasing element 320 enables foot massager 318 to move relative to foot base 60.

A pair of bands 322 and 324 are coupled to foot massager 318 and to leg massager 316. Specifically, bands 322 and 324 are coupled to the respective shaft 306 of foot massager 318 and leg massager 316. Accordingly, in the exemplary embodiment, foot massager 318 and leg massager 316 are tethered together such that motion of foot massager 318 causes motion of leg massager 316 via bands 322 and 324. Similarly, motion of leg massager 316 causes motion of foot massager 318. In the exemplary embodiment, bands 322 and 324 are substantially the same length (not shown) and are substantially inelastic, and each has a limited ability to stretch or elongate.

In the exemplary embodiment, massage system 300 includes a pair of medial pulleys 326 and a pair of lateral pulleys 328. Alternatively, system 300 may include more or less than two medial pulleys 326 and/or lateral pulleys 328. Each medial and lateral pulley 326 and 328 is rotationally coupled to device 10. First pair of medial pulleys 326 includes a first medial pulley 326a that is rotationally coupled to foot medial side 62, and a second medial pulley 326b that is rotationally coupled to medial side 46. Second pair of lateral pulleys 328 includes a first lateral pulley 328a that is rotationally coupled to foot lateral side 64 and a second lateral pulley (not visible) that is rotationally coupled to lateral side 50. First band 322 is operably engaged with each medial pulley 326a and 326b, and second band 324 is operably engaged with each lateral pulley 328.

Massage system 300 also includes at least one guide feature 330 that engages with first and second bands 322 and 324 to guide and align first and second bands 322 and 324, respectively. Guide features 330 may include eyelets or apertures that guide first and second bands 322 and 324, respectively. First band 322 is coupled to foot medial side 62 along a medial attachment location 336, and second band 324 is coupled to foot lateral side 64 along a lateral attachment location (not visible). Rotations of foot portion 14 that increase ankle angle α₉₀ causes first and second bands 322 and 324, respectively, to be pulled in a first direction against medial and lateral pulleys 326 and 328, respectively, thus moving foot massager 318 and leg massager 316 in a first direction in which foot massager 318 moves towards heel end 56, and leg massager 316 moves towards upper end 42. In contrast, rotations of foot portion 14 that decrease ankle angle α₉₀ causes first and second bands 322 and 324, respectively, to be pulled in a second direction against medial and lateral pulleys 326 and 328, respectively, thus moving foot massager 318 and leg massager 316 in a second direction in which foot massager 318 moves towards toe end 58, and leg massager 316 moves towards leg portion 12 lower end 44. Biasing element 320 may induce a force on foot massager 318 to bias foot massager 318 and leg massager 316 in second direction.

In some embodiments, a track or guide (not shown) is formed on foot portion 14 and/or leg portion 12, that is sized and shaped to engage with wheels 312 to guide the movement of massager rollers 302. Additionally, guide features 330 that engage first and second bands 322 and 324 to guide and align first and second bands 322 and 324, may also facilitate aligning the position and motion of the massage rollers 302.

With reference again to FIGS. 1-7, a user may wear device 10 by inserting their lower leg, foot, and toes within each of respective leg, foot, and toe cavities 24, 32, and 40. The user may adjust the fit and comfort of device 10, for example, by selectively inflating and/or deflating bags and/or selectively attaching/detaching cushions. A user may engage the muscles of their lower limb, for example their gastrocnemius and soleus muscles (not shown), to selectively position their foot and toes through a plurality of angles including either the plantarflexed and/or dorsiflexed positions. Accordingly, device 10 enables a user to control an amount of stretch of soft tissue structures of the lower limb, such as their plantar fascia, Achilles tendon, and/or other tendons, for example. Further, a user may selectively stretch and relax these structures to perform active stretching and to exercise musculature of the lower limb. Additionally, device 10 enables a user to exercise their lower limb while the tibiofemoral joint remains relatively unloaded (i.e., under minimal bodyweight forces). For example, a user may be seated while using device 10. Accordingly, device 10 may have particular advantages for users with additional musculoskeletal disorders, e.g., users with osteoarthritis, joint replacements, and/or muscle weakness.

In the exemplary embodiment, device 10 is aligned substantially symmetrically about axis A₁₂, axis A₁₄ and axis A₁₆ and as such, device 10 may be used on either the user's left leg or right leg. Additionally, and/or alternatively, device 10 may be shaped such that device 10 may be uniquely used for only left or right foot or leg of a user. For example, the foot medial side 62 of toe portion 16 may be extended and/or curved to accommodate toes of a user.

In some embodiments, various functional components such as, but not limited to, resistance system 100, nerve stimulation system 200, and massage system 300 may be selectively used or combined for use with device 10. These functional components may be used in isolation and/or in combination. Accordingly, device 10 may function in a modular manner, enabling a user to selectively attach/detach the functional components and to thus customize the device 10 to the user. For example, a user may selectively connect nerve stimulation system 200 and resistance system 100 thus enabling a user to perform active stretching exercises while simultaneously electrically stimulating a nerve of the user's foot or leg. Additionally, and/or alternatively, a user may selectively couple massage system 300 and resistance system 100, thus enabling a user to perform active stretching exercises while, simultaneously massaging the planter fascia and the Achilles tendon.

Compared to conventional treatments of musculoskeletal disorders of the lower limb, embodiments of the present disclosure have several advantages. The device enables the user to selectively position their foot and toes in a plurality of positions, including plantarflexed and/or dorsiflexed positions. For example, a user may activate the musculature of their lower limb to facilitate actively controlling the amount of stretch during use of device 10. In embodiments including resistance system 100, device 10 may be used to exercise and strengthen the muscles of the lower limb and to perform active stretching exercises. In embodiments including nerve stimulation system 200, device 10 supplies electrical stimulation to the lower leg and foot. In embodiments including massaging system 300, the massagers may be used to massage the lower leg (e.g., the Achilles tendon) and foot (e.g., Plantar fascia) of a user.

As used herein, the terms “about,” “substantially,” “essentially,” and “approximately” when used in conjunction with ranges of dimensions, concentrations, temperatures or other physical or chemical properties or characteristics is meant to cover variations that may exist in the upper and/or lower limits of the ranges of the properties or characteristics, including, for example, variations resulting from rounding, measurement methodology or other statistical variation.

When introducing elements of the present disclosure or the embodiment(s) thereof, the articles “a,” “an,” “the,” and “said” are intended to mean that there are one or more of the elements. The terms “comprising,” “including,” “containing,” and “having” are intended to be inclusive and mean that there may be additional elements other than the listed elements. The use of terms indicating a particular orientation (e.g., “top,” “bottom,” “side,” etc.) is for convenience of description and does not require any particular orientation of the item described.

As various changes could be made in the above constructions and methods without departing from the scope of the disclosure, it is intended that all matter contained in the above description and shown in the accompanying drawing[s] shall be interpreted as illustrative and not in a limiting sense.

What is claimed is:

1. An orthopedic device comprising:
a leg portion comprising a leg shell comprising an upper end and an opposite lower end, said leg shell at least partially defines a leg portion cavity that is sized to receive at least a portion of a user's lower leg therein, said leg portion having a leg axis extending between said upper and lower ends;
a foot portion comprising a foot shell comprising a heel end and an opposite toe end, said foot shell at least partially defines a foot portion cavity sized to receive at least a portion of the user's foot therein, said foot portion having a foot axis extending between said heel and toe ends, said foot portion is rotationally coupled to said leg portion at an ankle joint such that an ankle angle defined between the foot portion axis and the leg portion axis is selectively positionable in one of an acute ankle angle and an obtuse ankle angle; and
a toe portion comprising a toe shell comprising a first end and an opposite second end, said toe shell at least partially defines a toe portion cavity sized to receive at least a portion of the user's toes therein, said toe portion having a toe axis extending between said first and second ends, said toe portion is rotationally coupled to said foot portion along a toe joint such that a toe angle defined between the foot axis and toe axis is selectively positionable in one of an acute toe angle and an obtuse toe angle.

2. The orthopedic device of claim 1, wherein said device comprises at least one resistance band comprising a first end coupled to said leg portion and a second end coupled to said foot portion, said at least one resistance band resists rotation of at least one of said toe joint and said ankle joint.

3. The orthopedic device of claim 2, wherein said at least one resistance band comprises an elastic material that enables said resistance band to be selectively stretched from a first length to a second length that is longer than said first length, said resistance band selected such that said first length is defined when said ankle angle is at a first ankle angle, and such that said second length is defined when said ankle angle is at a second ankle angle that is greater than said first ankle angle.

4. The orthopedic device of claim 2, wherein said at least one resistance band has a band stiffness that is variably selectively adjustable via a knob, wherein rotating said knob in a first rotational direction increases the band stiffness and wherein rotating said knob in an opposite second rotational direction decreases the band stiffness.

5. The orthopedic device of claim 1 further comprising:
a massager biasedly coupled to at least one of said leg portion and said foot portion;
at least one pulley rotationally coupled to at least one of said leg portion and said foot portion; and
a band coupled to said at least one pulley, said band coupled to said massager and to at least one of said leg portion and said foot portion, wherein when said ankle angle is at a first ankle angle, said massager is in a first position, and when said ankle angle is moved to a second ankle angle, said massager is moved concurrently to a second position.

6. The orthopedic device of claim 5, wherein said massager comprises:
a foot massager positioned in close proximity to said foot portion; and
a leg massager coupled to said foot massager via said band, wherein when said ankle angle is at said first ankle angle, said leg and foot massagers are in said first position, and when said ankle angle is moved to said second ankle angle, said leg and foot massagers are moved concurrently to said second position.

7. The orthopedic device of claim 5, wherein said massager comprises an outer roller that is rotationally coupled to a shaft, said outer roller comprises a roller surface comprising a plurality of protrusions that extend outward from said outer roller to facilitate massaging at least one of the user's foot and leg.

8. The orthopedic device of claim 1, wherein said device comprises at least one transcutaneous electrical nerve stimulation (TENS) pad selectively coupled to at least one of said leg portion, said foot portion, and said toe portion.

9. The orthopedic device of claim 8, wherein said at least one TENS pad comprises a plurality of individual TENS units, each individual TENS unit is biasedly coupled to at least one of the said leg portion, said foot portion, and said toe portion.

10. The orthopedic device of claim 1 further comprising a locking mechanism used to secure a relative position of at least one of said ankle joint and said toe joint.

11. The orthopedic device of claim 1, wherein at least one of said leg shell, foot shell, and toe shell is selectively coupled to a cushion.

12. A system for exercising a user's lower limb, said system comprising:
a leg portion comprising a leg shell comprising an upper end and an opposite lower end, said leg shell at least partially defines a leg portion cavity that is sized to receive at least a portion of a user's lower leg therein, said leg portion having a leg axis extending between said upper and lower ends;
a foot portion comprising a foot shell comprising a heel end and an opposite toe end, said foot shell at least partially defines a foot portion cavity sized to receive at least a portion of the user's foot therein, said foot portion having a foot axis extending between said heel and toe ends, said foot portion is rotationally coupled to said leg portion at an ankle joint wherein an ankle angle defined between the foot axis and the leg axis is selectively positionable in a plurality of ankle angles such that said foot portion may be selectively positioned in one of dorsiflexed position and plantarflexed position with respect to said leg portion; and
a toe portion comprising a toe shell comprising a first end and an opposite second end, wherein said toe shell at least partially defines a toe portion cavity sized to receive at least a portion of the user's toes therein, said toe portion having a toe axis extending between said first and second ends, said toe portion is rotationally coupled to said foot portion at a toe joint wherein a toe angle defined between the foot axis and toe axis is selectively positionable in a plurality of toe angles such that said toe portion may be arranged in at least one of dorsiflexed position and plantarflexed position with respect to said foot portion.

13. The system of claim 12, wherein said system comprises at least one resistance band comprising a first end coupled to said leg portion and a second end coupled to said foot portion, said at least one resistance band resists rotation of at least one of said toe joint and said ankle joint.

14. The system of claim 13, wherein said at least one resistance band comprises an elastic material that enables said resistance band to be selectively stretched from a first length to a second length that is longer than said first length, said resistance band selected such that said first length is defined when said ankle angle is at a first ankle angle, and such that said second length is defined when said ankle angle is at a second ankle angle that is greater than said first ankle angle.

15. The system of claim 13, wherein said at least one resistance band has a band stiffness that is variably selectively adjustable via a knob, wherein rotating said knob in a first rotational direction increases the band stiffness and wherein rotating said knob in an opposite second rotational direction decreases the band stiffness.

16. The system of claim 12 further comprising:
a massager biasedly coupled to at least one of said leg portion and said foot portion;
at least one pulley rotationally coupled to at least one of said leg portion and said foot portion; and
a band coupled to said at least one pulley, said band coupled to said massager and to at least one of said leg portion and said foot portion, wherein when said ankle angle is at a first ankle angle, said massager is in a first position, and when said ankle angle is moved to a second ankle angle, said massager is moved concurrently to a second position.

17. The system of claim 16, wherein said massager comprises:
a foot massager positioned in close proximity to said foot portion; and
a leg massager coupled to said foot massager via said band, wherein when said ankle angle is at said first ankle angle, said leg and foot massagers are in said first position, and when said ankle angle is moved to said second ankle angle, said leg and foot massagers are moved concurrently to said second position.

18. The system of claim 12, wherein said system comprises at least one transcutaneous electrical nerve stimulation (TENS) pad selectively coupled to at least one of said leg portion, said foot portion, and said toe portion.

19. A method of fabricating an orthopedic device, said method comprising:
providing a leg portion that includes a leg shell having an upper end, an opposite lower end, and a leg portion axis extending between the upper and lower ends;
providing a foot portion including a foot shell including a heel end, an opposite toe end, and a foot portion axis extending between the heel and toe ends;
providing a toe portion that includes a toe shell including a first end, an opposite second end, and a toe axis extending between said first and second ends;
rotationally coupling the leg portion to the foot portion at an ankle joint such that an ankle angle defined between the foot portion axis and the leg portion axis is selectively positionable in one of an acute ankle angle and an obtuse ankle angle; and
rotationally coupling the toe portion to the leg portion at a toe joint such that a toe angle defined between the foot portion axis and toe portion axis is selectively positionable in one of an acute toe angle and an obtuse toe angle.

20. The method of claim 19 further comprising:
coupling a first end of at least one resistance band to the leg portion, said at least one resistance band resists rotation of at least one of the toe joint and the ankle joint; and
coupling a second end of the resistance band to the foot portion.
